(19) United States
(12) Patent Application Publication
(10) Pub. No.: US 2015/0045720 A1
(43) Pub. Date: Feb. 12, 2015

(54) METHOD FOR TREATING LOCAL INFECTION

(71) Applicant: A-Z LTD., Sendai-shi (JP)

(72) Inventors: Tarou Kanno, Sendai (JP); Keisuke Nakamura, Sendai (JP); Yoshimi Niwano, Osakasayama (JP); Minoru Kanno, Sendai (JP)

(21) Appl. No.: 14/452,999

(22) Filed: Aug. 6, 2014

Related U.S. Application Data
(60) Provisional application No. 61/863,489, filed on Aug. 8, 2013.

(51) Int. Cl. A61K 41/00; A61N 5/06

(52) U.S. Cl. CPC A61K 41/0057; A61N 5/062; A61N 2005/0662; A61N 2005/0661
USPC 604/20

(57) ABSTRACT

The present invention relates to a method for treating a local infection, for example, a periodontal disease, peri-implantitis or dermartitis, comprising applying a composition containing hydrogen peroxide and a polyphenol such as catechins to an infected site, for example, inside oral cavity or skin, and irradiating the site with light for a predetermined period of time.

### BACKGROUND OF THE INVENTION

(1) Field of the Invention

The present invention relates to a method for treating a local infection comprising applying a composition containing hydrogen peroxide and a polyphenol to an infected site and irradiating the site with light for a predetermined period of time.

(2) Description of Related Art

Hydrogen peroxide or hypochlorous acid is used in a method for disinfecting pathogenic bacteria with a local infection. However, hydrogen peroxide when used singly does not provide a sufficient disinfecting effect and hypochlorous acid is easily decomposed in the presence of an organic matter (Kishii et al., 2000a, 2000b; Mokudai et al., 2012), which are problematic.

To compensate these drawbacks, a disinfection method has been proposed which utilizes an intense oxidizing power of hydroxyl radical produced when hydrogen peroxide is irradiated with light having a wavelength of about 400 nm (Ikai et al., 2010). An advantage of this disinfection method is that since hydroxyl radical has an extremely short life, it quickly disappears when the light irradiation is halted, thereby minimizing the damage to the living body caused by hydroxyl radical. A method for enhancing the disinfecting activity of this technique is increasing the amount of hydroxyl radical to be produced by increasing a concentration of hydrogen peroxide or light irradiance. When practicing such a method, it is important to minimize the damage caused by hydroxyl radical produced during the light irradiation to the living body, and thus there is an enormous demand for a method which enhances the disinfecting activity while minimizing the damage to the living body.
1. Jiro Kishii, Mutsuo Yamauchi, Tooru Nagasawa: Effect of Denture Base Resin and Saliva Protein on Properties of Strong Acidic Electrolyzed Water; Part 1 Change of pH, Oxidation-Reduction Potential and Residual Chlorine Concentration, The Japanese Society for Dental Materials and Devices, 19, 27-33, 2000
2. Jiro Kishii, Mutsuo Yamauchi, Tooru Nagasawa: Effect of Denture Base Resin and Saliva Protein on Properties of Strong Acidic Electrolyzed Water Part 2 Change of Active Oxygen Species, The Japanese Society for Dental Materials and Devices, 19, 34-38, 2000
3. Mokudai, T., Nakamura, K., Kanno, T., Niwano, Y.: Presence of Hydrogen Peroxide, a Source of Hydroxyl Radicals, in Acid Electrolyzed Water. PLOS ONE, 7 (9):e46392, 2012
4. Ikai, H., Nakamura, K., Shirato, M., Kanno, T., Iwasawa, A., Sasakil, K., Niwano, Y., Kohno, M.: Photolysis of Hydrogen Peroxide, an Effective Disinfection System via Hydroxyl Radical Formation. Antimicrobial Agents and Chemotherapy, 54:5086-5091, 2010

### BRIEF SUMMARY OF THE INVENTION

On the other hand, JP-2011-011477 (WO2012/098772, U.S. patent application Ser. No. 13/807,224) (hereinafter referred to as Patent Literature 1) discloses that a far intenser disinfecting action can be achieved when a disinfecting agent containing catechins, which has already been well known to have a disinfecting action, is allowed to contact an object to be disinfected and irradiated with light.

Catechins, which are more stable and lower toxic than hydrogen peroxide, provide a stable disinfecting effect and are also very safe, compared with a disinfection method in which hydrogen peroxide is directly used.

Japanese Patent Application No. 2012-282607 discloses the use of a specific polyphenol to promote healing of wound, a wound-healing promoter containing a specific polyphenol, and a drug composition for treating wounds containing a specific polyphenol.

The present invention is based on a method for killing pathogenic microorganisms. In more detail, the present invention relates to, for example, a method for killing pathogenic microorganisms, which is capable of easily killing pathogenic microorganisms causing a local infection in skin, oral cavity or the like while assuring the safety to the human body. Specifically, the present invention proposes a photolysis disinfection method using hydrogen peroxide which enhances the infecting actions while minimizing the damages to the living body. The pathogenic microorganisms as used herein refer to the microorganisms which cause diseases such as fungi, bacteria and viruses.

In the treatment of oral cavity infections such as periodontal diseases, it is expected that an excellent therapeutic effect would be achieved by the combination of disinfecting causative microorganisms at an infected site and healing the wound.

The present inventors found that an unexpected remarkable synergistic disinfecting effect can be achieved by light irradiation while hydrogen peroxide and a polyphenol coexist (The Society for Antibacterial and Antifungal Agents, Japan, poster presentation at the 39th Annual General Meeting, Sep. 12, 2012; the article under submission to (Biocontrol Science)) and that an infected site can be thus disinfected safely and sufficiently. Furthermore, the inventors found that such an intense disinfecting effect disappears when the light irradiation is halted, which, after a predetermined period of the light irradiation, enables the quick transfer to the wound-healing process by a polyphenol while maintaining the sterilized state, whereby the present invention was accomplished.

### BRIEF DESCRIPTION OF THE DRAWINGS

FIG. 1 shows the disinfecting effect to S. mutans when laser light was irradiated to different concentrations of proanthocyanidin/hydrogen peroxide mixed solutions [mean+standard deviation (n=3)]. PA: Proanthocyanidin Significant differences from PA 0 mg/ml: P<0.05 (*), P<0.01 (**)

FIG. 2 shows the influence of proanthocyanidin on the disinfecting effect to P. gingivalis by the hydrogen peroxide photolysis disinfection method [mean+standard deviation (n=3)]. PA: Proanthocyanidin Significant differences between two groups: P<0.05 (*), P<0.01 (**)

FIG. 3 shows the influence of proanthocyanidin on hydroxyl radical produced by the hydrogen peroxide photolysis disinfection method (mean of duplicate). PA: Proanthocyanidin

FIG. 4 shows the concentrations of hydrogen peroxide produced when laser light was irradiated to the different concentrations of proanthocyanidin [mean+standard deviation (n=3)]. Left: laser light irradiation; Right: no laser light (shading)

### DETAILED DESCRIPTION OF THE PREFERRED EMBODIMENTS

Hereinafter, an embodiment of the present invention is described in detail.

An infected site is healed by the following steps. In the first step, a causative microorganism present at an infected site is disinfected by light irradiation in the presence of a composition containing hydrogen peroxide and a polyphenol. Then, simply halting the light irradiation triggers proceeding directly to the second step wherein the wound is healed by a polyphenol. The local infection is completely healed by the above two consecutive steps.

However, the above two steps do not need to be clearly distinguished and the wound healing is supposedly proceeding even in the above first step as disclosed in Japanese Patent Application No. 2012-282607 that “the effect of wound-healing promoter can be maintained even during the contact and even after irradiating the wounded site with ultraviolet laser light having a wavelength of 200 to 500 nm (preferably 405 nm).”

The polyphenols usable are those described in Patent Literature 1 and Japanese Patent Application No. 2012-282607 and examples include caffeine acid, catechins, chlorogenic acid, quercetin, rosmarinic acid, anthocyanidins such as cyanidin, delphinidin, aurantinidin, luteolinidin and petunidin, flavonoids such as cinchonine and quercetin, and polymers thereof.

Among the above polyphenols, caffeine acid, (+)-catechin and chlorogenic acid, and proanthocyanidin which is a polymer of catechins, are preferable. Proanthocyanidin is preferably an oligomer of (+)-catechin, (−)-epicatechin or (−)-epigallocatechin gallate.

In the treatment method according to the present invention, the composition may consist only of hydrogen peroxide and a polyphenol, or may contain other substances. The other substances may be water, saccharides, coloring agents, flavors, seasonings, synthetic or natural disinfecting agents other than polyphenols and hydrogen peroxide, and any other substances. Examples of the disinfecting agent other than polyphenols and hydrogen peroxide include strong acid water, iodine preparation (for example, tincture of iodine, povidone iodine), chlorines (for example, sodium hypochlorite), mercurochrome solution, chlorhexidine gluconate, acrinol, alcohols (for example, ethyl alcohol). However, the other substances are more preferably those which are high in safety.

According to the treatment method of the present invention, the concentration of hydrogen peroxide in the composition is preferably 80 to 500 mM, as suggested in Example below.

Similarly, the concentration of a polyphenol is preferably 1 to 8 mg/mL.

According to the treatment method of the present invention, the light may be that having any wavelength such as ultraviolet light and infrared light as long as it can produce hydroxyl radical but it is particularly preferable that the wavelength be 350 to 500 nm. This case also provides high safety as well as a high disinfecting effect. Particularly, when a visible light is used, higher safety can be achieved.

The irradiance of the light to be irradiated is preferably 300 mW/cm² or more. The larger the irradiance is, the greater the effectiveness is.

Furthermore, the irradiation time of the light is preferably 1 second to 10 minutes.

In the treatment method of the present invention, the method for applying the composition to an infected site may be any method such as coating or spraying the composition.

When applying the composition, the device described in U.S. 61/77842 and Japanese Patent Application No. 2013-139406 can be used.

### EXAMPLES

The present invention is described by way of the following example, but is not limited thereto.

The following test substances and reagents were subjected to a test. Leucoselect® manufactured by Indena (Milano, Italy) was used as proanthocyanidin. 4-Hydroxy-2,2,6,6-tetramethylpiperidine N-oxyl (TEMPOL) was purchased from Sigma Aldrich (St. Louis, USA), 5,5-dimethyl-1-pyrroline N-oxide (DMPO) was purchased from Labotec (Tokyo), hydrogen peroxide was purchased from Santoku Chemical Industries Co., Ltd. (Tokyo) and xylenol orange was purchased from Wako Pure Chemical Industries, Ltd. (Osaka), for use.

Proanthocyanidin was dissolved in phosphate buffered saline (PBS, pH 7.4) and adjusted to various concentrations, sterilized by filtration, and mixed with a hydrogen peroxide solution in different concentrations (hereinafter referred to as a proanthocyanidin/hydrogen peroxide mixed solution).

Streptococcus mutans JCM 5705 and Porphyromonas gingivalis JCM 12257 obtained from Riken BioResource Center (Wako city) were used as the test microorganisms. S. mutans was precultured on Brain Heart Infusion agar medium (Becton Dickinson Labware, Franklin lakes, USA) at 37° C. under anaerobic conditions. A microbial suspension (about 1×10⁸ cells/ml) was prepared from the precultured bacteria using PBS and subjected to the test. Then, 100 μl of the proanthocyanidin/hydrogen peroxide mixed solution in different concentrations and 100 μl of the microbial suspension were added to each well of a 96-well microplate and irradiated with laser light at a wavelength of 405±5 nm and an output power of 300 mW for 3 minutes (the irradiance was 930 mW/cm²). The laser light irradiation was carried out using a Ricoh Optical Industries Co., Ltd. (Hanamaki) laser device (RV-1000) equipped with an indium gallium nitride laser diode. After the laser light irradiation, an equivalent amount of a 5000 U/ml catalase solution (Wako Pure Chemical Industries, Ltd.) was added to 50 μl of the mixed solution to decompose the residual hydrogen peroxide and the reaction was halted. Then, 10 μl each of ten-fold serial dilution of this solution was sown onto the same agar medium as the preculture, cultured for 2 days, and the viable cell count was determined to calculate the colony forming units (CFU)/ml. P. gingivalis was precultured on Brucella agar medium (Kyokuto Pharmaceutical Industrial Co., Ltd., Tokyo) containing hemin supplemented with 5% horse blood and vitamin K at 37° C. under anaerobic conditions. A microbial suspension (about 1×10⁸ cells/ml) was prepared from the precultured bacteria using Difco (trade mark) Anaerobe Broth MIC (Becton Dickinson Labware) and subjected to the test. Proanthocyanidin was prepared and adjusted to a concentration of 2 mg/ml and hydrogen peroxide was prepared and adjusted to a concentration of 20 to 320 mM. Then, 100 μl of the proanthocyanidin/hydrogen peroxide mixed solution and 100 μl of the microbial suspension were added to each well of a 96-well microplate and irradiated with laser light for 30 seconds under the same conditions as above to calculate the CFU/ml by the culture test in the same manner as above.

Hydroxyl radical produced when the proanthocyanidin/hydrogen peroxide mixed solution was irradiated with laser light was analyzed as follows. First, 150 μl of the proanthocyanidin/hydrogen peroxide mixed solution in different solutions and 50 μl of DMPO were added to each well of a 96-well microplate and mixed (the final concentration of proanthocyanidin was 1 to 8 mg/ml, of hydrogen peroxide was 125 to 500 mM, and of DMPO was 445 mM). After 15 seconds of the laser light irradiation under the same conditions as above, the reaction solution was immediately transferred to a quartz cell and analyzed in an electron spin resonance (ESR) device (JES-FA-100; JEOL, Tokyo). The ESR measurement conditions are as below. Field sweep, 331.4-341.4 mT; field modulation frequency, 100 kHz; field modulation width, 0.1 mT; amplitude, 80; sweep time, 2 min; time constant, 0.03 s; microwave frequency, 9.420 GHz; microwave power, 4 mW. Used was 20 μM TEMPOL to be the standard substance, and the concentration of DMPO-OH, the spin adduct produced from hydroxyl radical and DMPO, was calculated.

The concentrations of hydrogen peroxide produced when the proanthocyanidin solution dissolved in PBS in different concentrations was and was not irradiated with laser light for 3 minutes were colorimetrically analyzed as follows. The analysis principle is the color development by the reaction of hydrogen peroxide-mediated oxidation of Fe²⁺ followed by the reaction of Fe³⁺ with xylenol orange. Specifically, 500 μl of the reaction solution (500 μM ammonium ferrous sulfate, 50 mM sulfuric acid, 200 μM xylenol orange and 200 mM sorbitol) was added to 500 μl of the proanthocyanidin solution, allowed to stand at room temperature for 45 minutes, and the absorbance at 560 nm was measured using a spectrophotometer (Gene Quant, GE Healthcare, Buckinghamshire, England).

The statistical analysis was carried out as follows. The viable cell count (CFU/ml) obtained by the bacterial test was logarithmically transformed. Then, the result for S. mutans was analyzed by Dunnett's multiple comparison test, and the result for P. gingivalis was analyzed by Student's t-test with a significant difference against the proanthocyanidin free control at a significance level of 5%.

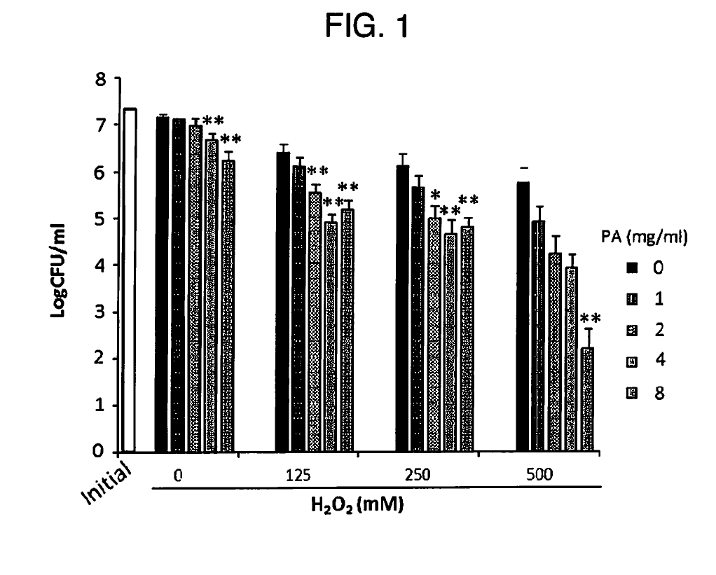

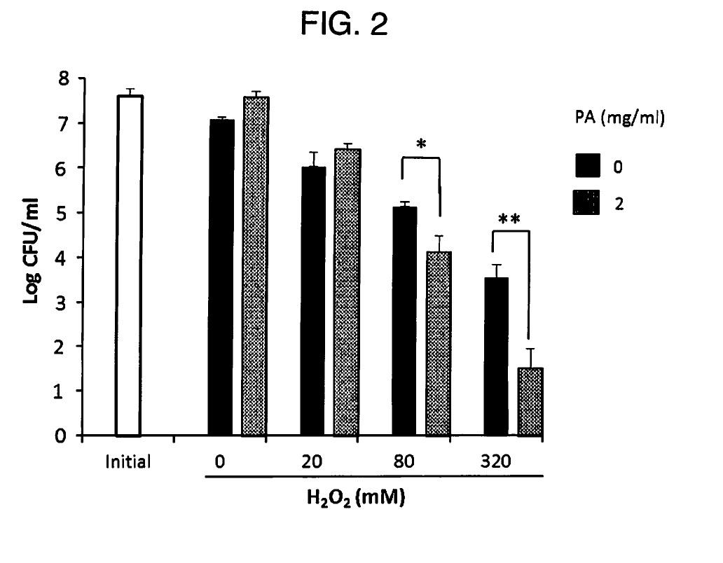

The disinfecting effects on S. mutans when the proanthocyanidin/hydrogen peroxide mixed solution in different concentrations was irradiated with laser light are shown in FIG. 1. The viable cell counts were reduced in a not only proanthocyanidin but also hydrogen peroxide concentration dependent manner. Particularly, with the combination of 500 mM hydrogen peroxide and 8 mg/ml proanthocyanidin, the viable cell count was reduced by about 5-log from the initial cell count, and synergistic disinfecting effect was thus found when compared with the individual effect provided by each of them. The results of disinfecting test on P. gingivalis conducted for the purpose of confirming such a synergistic effect are shown in FIG. 2. The addition of 2 mg/ml proanthocyanidin dramatically enhanced the disinfecting activity at the time of irradiating 80 and 320 mM hydrogen peroxide with laser light.

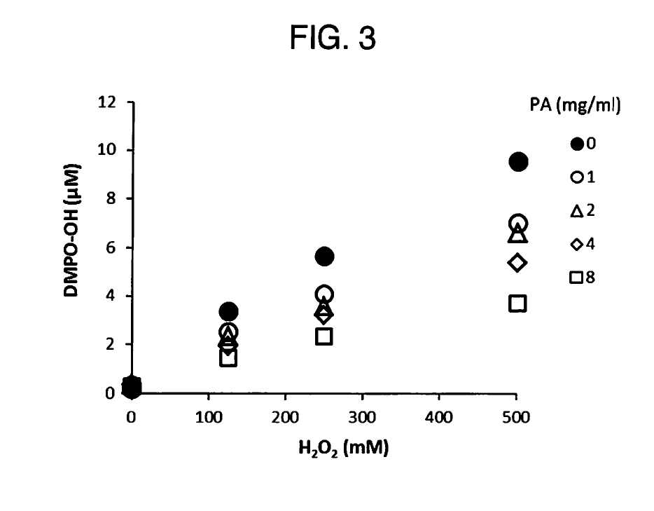

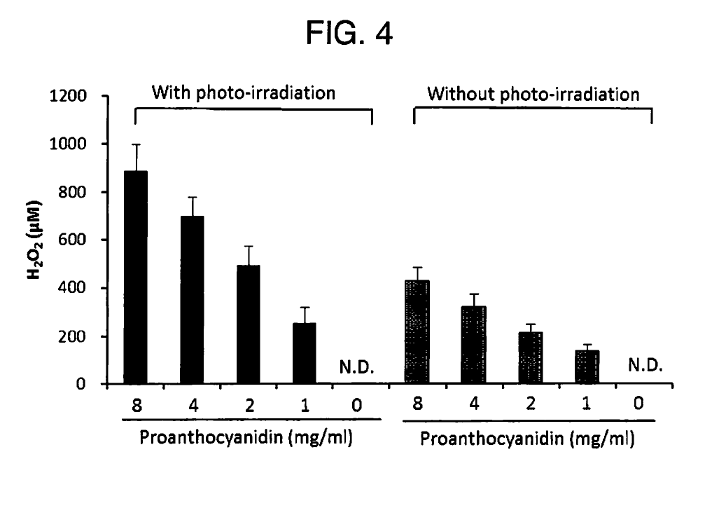

The disinfecting activity action of the photolysis disinfection method of hydrogen peroxide enhanced by the addition of proanthocyanidin is hypothetically attributable to hydroxyl radical, which is obtained by further photolyzing hydrogen peroxide produced when dissolved oxygen is reduced by the proton and electron released from the photooxidized phenolic hydroxyl group of proanthocyanidin, and accordingly hydroxyl radical was analyzed by ESR. The results are shown in FIG. 3. The hydroxyl radical amount represented by DMPO-OH increased in a hydrogen peroxide concentration dependent manner, but the proanthocyanidin addition unexpectedly reduced hydroxyl radical amount in a concentration dependent manner. Then, the production of hydrogen peroxide was investigated next, since the hypothesis was that hydrogen peroxide produced as a result of photooxidized proanthocyanidin was the source of hydroxyl radical. The results are shown in FIG. 4. It is revealed that when proanthocyanidin is irradiated with laser light, hydrogen peroxide is produced in a proanthocyanidin concentration dependent manner. The production of hydrogen peroxide was also observed in a proanthocyanidin solution under the condition with no laser light irradiation, though the concentration of hydrogen peroxide was low.

According to the above, the following is strongly suggested: a part of hydroxyl radical in the solution produced by the photolysis of hydrogen peroxide disappears by the antioxidative action of proanthocyanidin, but reactive oxygen species, such as hydrogen peroxide and hydroxyl radical, produced via the photooxidation of proanthocyanidin adsorbed onto a bacterial cell oxidatively damage the bacterial cell at the local surface thereof; and thereby hydrogen peroxide more easily penetrates into the bacterial cell and is photolyzed therein to produce hydroxyl radical, which effectively works only locally, exhibiting the synergistic effect. Such a result dramatically demolishes the conventional belief that an antioxidant such as proanthocyanidin capable of scavenging the reactive oxygen species such as hydroxyl radical attenuates the effect of photolysis disinfection method using hydrogen peroxide. The present invention is based on such a result and therefore involves an inventive step of enhancing the disinfecting activity while minimizing the damage to the living body over a prior art of the photolysis disinfection method using hydrogen peroxide.

1. A method for treating a local infection comprising:
applying a composition containing hydrogen peroxide and a polyphenol to an infected site, and
irradiating the infected site with light for a predetermined period of time.

2. The method according to claim 1, wherein the infected site is inside oral cavity or skin.

3. The method according to claim 2, wherein the local infection is a periodontal disease, peri-implantitis or dermatitis.

4. The method according to claim 1, wherein the polyphenol is catechins.

5. The method according to claim 4, wherein the catechins are proanthocyanidin.

6. The method according to claim 1, wherein the composition is an aqueous solution.

7. The method according to claim 6, wherein a concentration of the hydrogen peroxide is 80 to 500 mM.

8. The method according to claim 6, wherein a concentration of the polyphenol is 1 to 8 mg/mL.

9. The method according to claim 1, wherein a wavelength of the light is 350 to 500 nm.

10. The method according to claim 1, wherein the light is a visible light.

11. The method according to claim 9, wherein the wavelength is 400±5 nm

12. The method according to claim 1, wherein an irradiation time of the light is 1 second to 10 minutes.
